(19) United States
(12) Patent Application Publication
(10) Pub. No.: US 2025/0208339 A1
(43) Pub. Date: Jun. 26, 2025

(54) PLASTIC OPTICAL FIBER AND MEDICAL SENSOR DEVICE

(71) Applicant: Toray Industries, Inc., Tokyo (JP)

(72) Inventors: Masahiro Yoshioka, Nagoya-shi, Aichi (JP); Hidekazu Kunieda, Tokyo (JP); Kana Yamazaki, Tokyo (JP); Kenichi Kawabe, Nagoya-shi, Aichi (JP)

(73) Assignee: Toray Industries, Inc., Tokyo (JP)

(21) Appl. No.: 18/848,308

(22) PCT Filed: Mar. 23, 2023
(86) PCT No.: PCT/JP2023/011410

§ 371 Date: Sep. 18, 2024

(30) Foreign Application Priority Data
Mar. 31, 2022 (JP) 2022-058164

(51) Int. Cl. G02B 6/036; G02B 6/028

(52) U.S. Cl. CPC G02B 6/036; G02B 6/028

(57) ABSTRACT

A plastic optical fiber has an excellent noise suppression effect. The plastic optical fiber is a plastic optical fiber including a core and a cladding, wherein a first cladding adjacent to the core contains a light-shielding agent, and a content of the light-shielding agent is 100 to 10000 ppm, the plastic optical fiber preferably further includes a second cladding outside the first cladding. In addition, the plastic optical fiber can be suitably used for a medical sensor device.

### CROSS-REFERENCE TO RELATED APPLICATIONS

This application is a US national stage filing under 35 U.S.C. § 371 of International Application No. PCT/JP2023/011410, filed Mar. 23, 2023, which claims priority to Japanese Patent Application No. 2022-058164, filed Mar. 31, 2022, each of which is incorporated herein by reference in its entirety.

### TECHNICAL FIELD

This disclosure relates to a plastic optical fiber and a medical sensor device.

### BACKGROUND

Plastic optical fibers are superior to glass optical fibers in terms of processability, handleability, manufacturing cost, and the like, and are therefore used for short-distance optical signal transmission, light guide, sensor applications, and the like.

A plastic optical fiber is usually composed of two layers of a core and a first cladding, and a polymer having excellent transparency and favorable weatherability, as represented by polymethyl methacrylate (hereinafter, abbreviated as PMMA), is typically used for the core. On the other hand, the cladding is required to have a refractive index lower than that of the core to confine light inside the core, and a fluorine-containing polymer is widely used. The condition of occurrence of total reflection of light inside the core is that a propagation angle θ is smaller than a critical angle (total reflection angle) θ₀. The critical angle (total reflection angle) θ₀ is obtained by Sinθ₀=n2/n1, where n1 is the refractive index of the core and n2 is the refractive index of the cladding. On the other hand, light larger than the critical angle θ₀ enters the cladding. The light entering the cladding is totally reflected in a case where the propagation angle is less than a critical angle (total reflection angle) θ₁ obtained by Sinθ₁=n3/n2, where n2 is the refractive index of the cladding and n3 is the refractive index of the air. Since the core refractive index n1 is larger than the cladding refractive index n2, the totally reflected light has no critical angle and returns to the core again. In general, in a case where the optical fiber is used in a sufficiently long state for communication applications or the like, the light returning to the core through the cladding becomes light having the critical angle (total reflection angle) θ₁ or more by light scattering in the cladding or bending of the fiber, enters the air layer, and gradually attenuates, causing no major problem. However, in the case of use with a length of several meters or less for sensor applications or the like, there is the problem that the light propagating through the cladding is detected without attenuation, and becomes noise. As a plastic optical fiber with reduced noise, an optical fiber has been proposed in which a resin layer having a refractive index higher than that of a first cladding layer adjacent to a core is formed as a second cladding layer (see, for example, Japanese Patent Laid-open Publication No. 2003-227976).

In addition, it has been proposed to add a light-shielding agent to a second cladding (see, for example, International Publication No. 2022/009653).

Furthermore, it has been proposed that the refractive index of a first cladding is larger than that of a second cladding, and a light-shielding agent is added to the second cladding (see, for example, Japanese Patent Laid-open Publication No. 2002-98864).

However, in the method described in Japanese Patent Laid-open Publication No. 2003-227976, the refractive index of the second cladding is larger than the refractive index of the first cladding, so that the critical angle (total reflection angle) between the first cladding and the second cladding disappears, and light propagation through the first cladding is reduced. However, there is the problem that a new light propagation route through the second cladding is generated by the second cladding being sandwiched between the first cladding and the air layer that have refractive indexes lower than that of the second cladding.

In addition, in the method described in International Publication No. 2022/009653, the refractive index of the second cladding is smaller than the refractive index of the first cladding. Therefore, there is a critical angle (total reflection angle) at an interface between the second cladding and the first cladding. Light larger than the critical angle enters the second cladding. Since the light-shielding agent is contained in the second cladding, the light entering the second cladding gradually attenuates. Light smaller than the critical angle is partly absorbed by the light-shielding agent on the second cladding surface, but is mostly totally reflected.

Furthermore, in the method described in Japanese Patent Laid-open Publication No. 2002-98864, the refractive index of the second cladding is larger than the refractive index of the first cladding, so that the critical angle (total reflection angle) between the first cladding and the second cladding disappears, light propagation through the first cladding is reduced, and light entering the second cladding gradually attenuates. However, even in that method, noise has not been eliminated. This suggests the following fact. In general, in a case where a fiber is formed by melt spinning, there is a compatible layer of several tens nm to several hundreds nm between layers. Thus, even when the refractive index of the second cladding is larger than the refractive index of the first cladding, light does not completely enter the second cladding from the first cladding, and there is light reflected between the second cladding and the first cladding.

It could therefore be helpful to provide a plastic optical fiber and a medical sensor device having an excellent effect of suppressing noise generated by light propagation through a cladding in the plastic optical fiber.

### SUMMARY

Disclosed herein is:

(1) A plastic optical fiber including a core and a cladding, in which a first cladding adjacent to the core contains a light-shielding agent, and a content of the light-shielding agent is 100 to 10000 ppm.
(2) The plastic optical fiber according to (1), in which the light-shielding agent contained in the first cladding is carbon black.
(3) The plastic optical fiber according to (1) or (2), further including a second cladding outside the first cladding.
(4) The plastic optical fiber according to (3), in which a refractive index of the second cladding is larger than a refractive index of the first cladding.
(5) The plastic optical fiber according to (3) or (4), in which the second cladding contains a light-shielding agent, and a content of the light-shielding agent is 100 to 10000 ppm.
(6) The plastic optical fiber according to (5), in which a transmittance t1 of light having a wavelength of 650 nm at a film thickness of the first cladding and a transmittance t2 of light having a wavelength of 650 nm at a film thickness of the second cladding have a relationship of t1>t2.
(7) The plastic optical fiber according to (5) or (6), in which the light-shielding agent contained in the second cladding is carbon black.
(8) The plastic optical fiber according to any one of (5) to (7), in which the light-shielding agent contained in the first cladding and the light-shielding agent contained in the second cladding are made of the same material, and a light-shielding agent content D1 of the first cladding and a light-shielding agent content D2 of the second cladding have a relationship of D1<D2.
(9) The plastic optical fiber according to any one of (4) to (8), in which a plastic optical fiber forming film thickness T1 of the first cladding and a plastic optical fiber forming film thickness T2 of the second cladding have a relationship of T1<T2.
(10) The plastic optical fiber according to any one of (1) to (9), in which the core is made of polymethyl methacrylate, and a numerical aperture NA of the core and the first cladding is 0.65 or less.
(11) A medical sensor device including the plastic optical fiber according to any one of (1) to (10).

It is thus possible to provide a plastic optical fiber having an excellent effect of suppressing noise generated through a cladding. This noise suppression effect also exerts an excellent effect in a conventional plastic optical fiber that is used with a length of several meters or less where light propagation through a cladding cannot be attenuated to become noise as in a sensor.

Therefore, it is possible to provide a plastic optical fiber suitable for industrial sensors for a semiconductor manufacturing apparatus, an automobile manufacturing apparatus, and the like, or medical sensors for blood oxygen concentration measurement or the like.

### DETAILED DESCRIPTION

The following specifically describes embodiments of a plastic optical fiber and a plastic optical fiber cord including the same. This disclosure is not limited to the following embodiments and can be embodied in a variously modified manner in accordance with objects and uses.

A plastic optical fiber according to an embodiment includes a core and a first cladding adjacent to the core in this order. The first cladding is provided to be adjacent to the core and cover the periphery of the core. A second cladding layer may be provided outside the first cladding layer, and a jacket layer may be provided outside the first cladding layer or further outside the second cladding layer provided outside the first cladding layer as necessary. It is preferable that a core layer, the first cladding layer, and the second cladding layer and the jacket layer provided as necessary are substantially concentric with each other from the viewpoint of optical axis centering at the time of sensor connector connection.

In a case where the plastic optical fiber is used as a multi-core fiber, the first cladding adjacent to the core preferably has a uniform thickness. Specifically, in a method for measuring the thickness of the first cladding described later, it is preferable to adopt a shape that allows a circle to be drawn by extracting any three points of the first cladding at an interface between the first cladding and the air (or the second cladding, or the jacket layer described later), that is, any three points on the outer periphery of the first cladding (three points at which the central angle of an arc is 120°±10°), and passing through the three points. Half of a value obtained by subtracting the diameter of the core from the diameter of the drawn circle is the thickness of the first cladding. The core diameter is measured by a method described later. In a case where the second cladding is provided outside the first cladding, and in the case of a three-layer single core, it is preferable that the second cladding also has a uniform thickness. Specifically, it is preferable to adopt a shape that allows a circle to be drawn by extracting any three points at an interface between the second cladding and the air (or the jacket layer described later), that is, any three points on the outer periphery of the second cladding (three points at which the central angle of an arc is 120°±10°). In addition, a three-layer multi-core may be adopted in which the second cladding layer exists as a sea state outside the first cladding layer. In this case, the thickness of the second cladding layer is defined as the thickness of a thinnest portion.

### Core

A core material of the plastic optical fiber is preferably a (co)polymer containing methyl methacrylate (hereinafter, sometimes abbreviated as MMA) as a main component of a copolymerization component from the viewpoint of transmittance. Specifically, the (co)polymer includes a copolymer in which polymethyl methacrylate (hereinafter, sometimes abbreviated as PMMA) or MMA is 70 wt % or more of the copolymerization component. Examples thereof include modified polymers such as glutaric anhydride and glutarimide obtained by copolymerizing (meth)acrylic ester, (meth)acrylic acid, (substituted) styrene, (N-substituted) maleimide, and the like, or subjecting these to a polymer reaction. The above (co)polymer represents a polymer and a copolymer. Similarly, the (meth)acrylic ester represents an acrylic ester and a methacrylic ester. Examples of the (meth)acrylic ester include methyl acrylate, ethyl acrylate, ethyl methacrylate, butyl methacrylate, t-butyl methacrylate, cyclohexyl methacrylate, benzyl methacrylate, phenyl methacrylate, bornyl methacrylate, and adamantyl methacrylate. Examples of the substituted styrene include methylstyrene and a-methylstyrene. Examples of the N-substituted maleimide include N-isopropylmaleimide, N-cyclohexylmaleimide, N-methylmaleimide, N-ethylmaleimide, and N-o-methylphenylmaleimide. A plurality of these copolymerization components may be used, and a small amount of component other than these components may be used. In addition, a stabilizer such as an antioxidant may be contained in an amount that does not adversely affect transparency.

As another polymer forming the core, for example, a cycloolefin polymer (COP), a cycloolefin copolymer (COC), polystyrene, polycarbonate, fluorene-containing polyester, polymethylpentene, and the like can also be preferably used as long as a fiber numerical aperture (hereinafter, NA) does not exceed 0.65 in combination of the core and the first cladding. When the NA exceeds 0.65, noise (hereinafter, S/N ratio) tends to increase in the plastic optical fiber. The S/N ratio refers to a value obtained by dividing a light intensity distribution emitted from the fiber into an area of θm/10 to the inside from a one-side maximum light emission angle θm calculated from the fiber numerical aperture (hereinafter, NA) and an area of a maximum light emission angle ±θm/10, and dividing a maximum light intensity (S) in the area of θm/10 to the inside from the maximum light emission angle by a maximum light intensity (N) in the area of the maximum light emission angle ±θm/10. For example, when the maximum light emission angle is 40°, the S/N ratio is calculated as a value obtained by dividing the maximum light intensity (S) in an area of 4° to the inside from the maximum light emission angle, that is, an area of 0 to 36° by the maximum light intensity (N) in an area of 36 to 44°.

The core diameter of the plastic optical fiber is preferably 100 to 3000 μm. The core diameter of 100 μm or more is preferable in that a sufficient amount of light can be obtained as the plastic optical fiber. In addition, the core diameter of 3000 μm or less is preferable in that the plastic optical fiber has a size suitable for a sensor.

The amount of light here refers to the transmission loss (dB/km) of the optical fiber calculated by a cutback method.

### First Cladding

The plastic optical fiber includes the cladding in addition to the core, and the cladding adjacent to the core is referred to as first cladding. In a case where the core material is PMMA (refractive index: 1.48 to 1.50), a cladding material used for the first cladding is preferably a low-refractive fluororesin from the viewpoint of NA. The fluororesin is not particularly limited, but it is preferable to use a copolymer having a vinylidene fluoride unit and a trifluoroethylene unit (refractive index: 1.39 to 1.41), a fluorinated acrylic ester polymer having a refractive index lower than that of the copolymer, good adhesion to PMMA, and excellent processability (refractive index: 1.35 to 1.37), polyperfluorobutyl methacrylate (refractive index: 1.36), polyperfluoroisopropyl methacrylate (refractive index: 1.37), and polyhexafluoro2-propyl methacrylate (refractive index: 1.38). In addition, a dopant such as a fluorine-based material such as magnesium fluoride may be added for the purpose of further lowering the refractive index.

As another material forming the cladding, for example, in a case where the core is a cycloolefin polymer (COP), a cycloolefin copolymer (COC), polystyrene, polycarbonate, fluorene-containing polyester, polymethylpentene, and the like, PMMA can also be used for the cladding material as a resin other than the fluororesin as long as the NA does not exceed 0.65 in combination with the cladding. When the core material is PMMA having a refractive index of about 1.49, a refractive index difference between the core and the cladding is preferably 0.03 to 0.15. In addition, when the core material is a cycloolefin polymer having a refractive index of about 1.54, the refractive index difference between the core and the cladding is preferably 0.03 to 0.15. In addition, when the core material is fluorene-containing polyester having a refractive index of about 1.64, the difference is preferably 0.03 to 0.14.

The first cladding of the plastic optical fiber is characterized by containing a light-shielding agent. As the light-shielding agent, both of an organic pigment and an inorganic pigment can be used. For example, carbon black, titanium black, and the like can be used. Among them, carbon black is preferably used. Light to be shielded is assumed to have a wavelength of 650 to 1000 nm, which is typically used as a sensor, but is not necessarily limited thereto. By containing the light-shielding agent, noise by light propagation through the first cladding can be suppressed.

In the plastic optical fiber, the first cladding adjacent to the core preferably contains carbon black as the light-shielding agent. The content of the light-shielding agent such as carbon black in the cladding material is preferably 100 to 10000 ppm. When the content of the light-shielding agent such as carbon black is 100 ppm or more, detection of noise is suppressed, and when the content is 10000 ppm or less, a sufficient amount of light emitted from the core can be obtained.

The content of the carbon black contained in the cladding of the optical fiber can be identified by SEM observation. The SEM observation of the cross section of the optical fiber enables the resin (cladding material) forming the cladding and the carbon black to be distinguished from each other, whereby an area ratio between the resin forming the cladding and the carbon black can be calculated. When this area is multiplied by the specific gravity of each material, a weight ratio can be calculated. From the viewpoint of variation, it is preferable to prepare SEM samples having different cross sections and calculate the measurement average of 10 samples. The content identification method can be applied not only to the carbon black but also to the organic pigment, the inorganic pigment, and the like.

The first cladding thickness of the plastic optical fiber is preferably 5 to 50 μm.

When the thickness is 5 μm or more, the first cladding has a sufficient function as the cladding. In addition, when the thickness is 50 μm or less, the first cladding does not have an excessive thickness as the cladding and has a preferable size.

### Second Cladding

The plastic optical fiber may further include the second cladding outside the first cladding. In a case where the second cladding is provided, the refractive index of the second cladding is preferably larger than the refractive index of the first cladding. As a result, in addition to a function of protecting the first cladding, the second cladding can be provided with a function of suppressing light propagation through the first cladding by eliminating reflection at an interface between the first cladding and the second cladding and releasing light in the first cladding layer to the second cladding because of the refractive index larger than that of the first cladding layer. However, as described above, a compatible layer of several tens nm to several hundreds nm formed by melt spinning is present between the first cladding and the second cladding. Thus, not 100% of light enters the second cladding layer from the first cladding. The refractive index of a resin forming the second cladding only needs to be larger than that of the resin forming the first cladding, and the value is not particularly specified. However, in a case where the first cladding is a fluororesin, a resin such as a fluororesin having a refractive index larger than the refractive index of the fluororesin of the first cladding, or PMMA can be used. In addition, in a case where the first cladding is a PMMA resin, a resin such as cycloolefin or polycarbonate having a refractive index larger than the refractive index of the PMMA can be used.

A refractive index difference between the second cladding and the first cladding is preferably 0.01 or more although there is no problem in principle as long as the second cladding has a larger refractive index. This is because the third decimal place of the refractive index varies depending on a raw material lot, and there is a possibility that the refractive index difference disappears.

The second cladding of the plastic optical fiber preferably contains a light-shielding agent. As the light-shielding agent, both of an organic pigment and an inorganic pigment can be used. For example, carbon black, titanium black, and the like can be used. Among them, carbon black is preferably used.

Of the light entering the second cladding, light smaller than a critical angle between the second cladding and the air is reflected at an air interface having a refractive index lower than that of the second cladding. Furthermore, of the reflected light, light smaller than a critical angle between the first cladding and the second cladding is reflected again at a first cladding interface having a refractive index lower than that of the second cladding. In this manner, noise due to light propagation through the cladding may also occur in the second cladding. Therefore, the light-shielding agent such as carbon black can serve to absorb such light. Examples of the usable light-shielding agent include those similar to the exemplified light-shielding agent contained in the first cladding. An addition amount of the light-shielding agent such as carbon black is preferably 100 to 10000 ppm. When the addition amount is 10000 ppm or less, the light-shielding agent such as carbon black has favorable dispersibility, and the fiber diameter of the plastic optical fiber can be favorably maintained.

It is preferable that the light-shielding agent contained in the first cladding and the light-shielding agent contained in the second cladding of the plastic optical fiber are made of the same material, and a light-shielding agent content D1 of the first cladding and a light-shielding agent content D2 of the second cladding have a relationship of D1<D2. When the carbon black content D1 of the first cladding is large, a trade-off occurs in which the noise suppression effect is improved, but the amount of emitted light is reduced due to absorption by the carbon black. Therefore, the carbon black content D2 of the second cladding is made larger than D1, which makes it easier to achieve both the noise suppression effect and the amount of emitted light. In addition, the second cladding also serves to block ambient light from the outside. Thus, the higher the concentration, the better the ambient light blocking property.

In addition, in the plastic optical fiber, a plastic optical fiber forming film thickness T1 of the first cladding and a plastic optical fiber forming film thickness T2 of the second cladding preferably have a relationship of T1<T2. When the film thickness T1 of the first cladding is thick, light scattering in the first cladding increases a light amount loss. Thus, in a case where the second cladding is present, it is more effective to absorb much noise in the second cladding by reducing the film thickness of the first cladding as much as possible and increasing the film thickness of the second cladding.

In addition, in the plastic optical fiber, a transmittance t1 of light having a wavelength of 650 nm at the film thickness of the first cladding and a transmittance t2 of light having a wavelength of 650 nm at the film thickness of the second cladding preferably have a relationship of t1>t2.

The transmittance at the film thickness of the cladding refers to a transmittance in a case where each material used for the cladding is formed into a film having the same film thickness as the film thickness of the cladding. In practice, it is difficult to produce a film having the same thickness as the film thickness of the cladding. Thus, a film of 100 μm is formed, and the transmittance is measured with a spectrophotometer. From the transmittance at the film thickness of 100 μm, the transmittance at the film thickness of the cladding is calculated. When the transmittance t1 of light having a wavelength of 650 nm at the film thickness of the first cladding is low, a trade-off occurs in which the noise suppression effect is improved, but the amount of emitted light is reduced due to absorption by the light-shielding agent. Therefore, the transmittance t2 of light having a wavelength of 650 nm at the film thickness of the second cladding is made lower than t1, which makes it easier to achieve both the noise suppression effect and the amount of emitted light. In addition, the second cladding also serves to block ambient light from the outside. Thus, the lower the transmittance, the better the ambient light blocking property.

To directly measure the transmittance of the cladding of the optical fiber, for example, the following method can be used. That is, the optical fiber is embedded in an epoxy resin to collect a measurement sample. For this measurement sample, a cross section in a direction perpendicular to the longitudinal direction of the optical fiber is polished until the length in the longitudinal direction of the optical fiber becomes 100 μm, and a light beam having a wavelength of 650 nm is emitted in the longitudinal direction from the cross section using high-resolution microspectroscopy to measure the transmittance of a cladding portion. As a result, the transmittance of the cladding portion having a thickness of 100 μm is obtained. The transmittance at the cladding thickness is calculated from the obtained transmittance at the thickness of 100 μm.

### Core Material

In addition, the core material of the plastic optical fiber is most preferably polymethyl methacrylate, and the numerical aperture NA of the core and the first cladding is most preferably 0.65 or less. While our fibers are intended to suppress noise by containing the light-shielding agent in the cladding, there is a concern that transmission loss may increase due to light absorption by the light-shielding agent, and thus polymethyl methacrylate having the highest transmittance is preferable as the core material. In addition, from the viewpoint of noise suppression, when the numerical aperture exceeds 0.65, that is, when the spread of light exceeds ±40.5°, a region for determining noise is widened. Thus, the NA is preferably 0.65 or less.

The type and amount of the light-shielding agent and the film thickness of each cladding may be adjusted according to the degree of noise suppression required. These means may also be adjusted in combination.

### Method for Producing Plastic Optical Fiber

As a method for producing the plastic optical fiber, for example, conjugate spinning is preferably used in which a raw material for forming the core material, a raw material for forming the first cladding material, and a material for forming the second cladding material as an optional component are individually sufficiently dried under a heating vacuum of 50 to 90° C., and then discharged from a conjugate spinneret for concentric conjugation under a heated and molten state of 200 to 300° C. to form a two-layer or three-layer core-sheath structure of the core/first cladding/second cladding (optional component). Subsequently, for the purpose of improving mechanical properties such as strength at break, drawing of about 1.2 to 3 times is typically performed to form the plastic optical fiber.

### Jacket

The plastic optical fiber according to the embodiment may include at least one jacket layer on the outer layer of the plastic optical fiber described above. Examples of a material forming the jacket layer include polyethylene, polypropylene, or copolymers thereof, blended products, olefin-based polymers having an organic silane group, ethylene-vinyl acetate, polyvinyl chloride, polyvinylidene fluoride, polyamide resins such as nylon 12, polyester resins, nylon elastomers, polyester elastomers, urethane resins, fluororesins, and rubbers such as EPM and EPDM. The jacket layer may be a single layer or multiple layers. In the case of multiple layers, a tension member such as “Kevlar” (registered trademark) may be placed between the jacket layers. These jacket layers may contain an antioxidant, an anti-aging agent, a stabilizer such as a UV stabilizer, and the like in addition to or in place of a flame retardant. The jacket layer can be preferably formed by a normal method such as melt extrusion molding using a cross-head die after first forming the plastic optical fiber by conjugate melt spinning.

### Plastic Optical Fiber

The plastic optical fiber thus obtained has an excellent effect of suppressing noise propagating through the cladding. As described above, even the plastic optical fiber not having a sufficient length for attenuating the noise, for example, having a length of several meters or less has an excellent effect of suppressing noise generated by light propagation through the cladding, and thus can be suitably used as a plastic optical fiber for industrial sensors for a semiconductor manufacturing apparatus, an automobile manufacturing apparatus, and the like, or medical sensors for blood oxygen concentration measurement or the like.

### EXAMPLES

Hereinafter, our fibers and devices are described in more detail by way of examples. Evaluations in Examples and Comparative Examples were performed by the following methods.

### (1) Core Diameter/First Cladding Thickness/Second Cladding Thickness

The core diameter/first cladding thickness/second cladding thickness can be measured by cutting five places randomly selected from the plastic optical fiber perpendicularly to the drawing direction, polishing the cross section so that the interface of the core/first cladding/second cladding can be observed, and then performing enlarged observation using a digital microscope VHX-7000 (manufactured by Keyence Corporation). The magnification of the enlarged observation is selected from the range of 10 to 200 times where the entire cross section falls within a visual field range and the interface can be observed. In the cross section, the diameter of a circle drawn by extracting three points so that the central angle of each arc was 120°±10° on a boundary circle formed by the interface between the core and the first cladding and passing through the three points was defined as the core diameter. Half of a value obtained by subtracting the core diameter from the diameter of a circle drawn by extracting any three points (three points at which the central angle of an arc falls within 120°±10°) of the first cladding at the interface between the first cladding and the air (or the second cladding) was defined as the first cladding thickness. Half of a value obtained by subtracting the cladding diameter from the diameter of a circle drawn by extracting any three points (three points at which the central angle of an arc falls within 120°±10°) of the interface between the second cladding and the air was defined as the second cladding thickness.

### (2) Refractive Index

A test piece of 10 mm×10 mm×3 mm was prepared from a material used in each of Examples and Comparative Examples by injection molding at 250° C., and the refractive index was measured in an atmosphere at room temperature of 25° C. using an Abbe refractometer.

### (3) Numerical Aperture NA

From the refractive index measured by the above-described method, the numerical aperture was calculated by the following formula.

Numerical aperture of core/first cladding=((refractive index of core)²−(refractive index of first cladding)²)^{1/2}.

### (4) Transmission Loss of Plastic Optical Fiber

By using a sample obtained by cutting the spooled plastic optical fiber obtained in each of Examples and Comparative Examples to a length of 5 m, collimated light (wavelength: 650 nm, incident NA=0.25) of a halogen lamp is incident from one end, and an amount of light A (dBm) emitted from the other end is measured. Subsequently, this 5 m sample was cut to a length of 2 m, the same collimated light was incident from one end, an amount of light B (dBm) emitted from the other end was measured, and a transmission loss C (dB/km) was obtained from (B−A)/(5−2).

### (5) Noise S/N Ratio

The spooled plastic optical fiber obtained in each of Examples and Comparative Examples was cut to a length of 2 m, and laser light (wavelength: 650 nm, incident NA=0.65) was incident from the end. The spot diameter at this time was adjusted to be the fiber diameter. The light emitted from one fiber end was applied to a screen of Lase View-LHB (manufactured by Kokyo, Inc) from a position of 2.5 mm, to measure the light intensity distribution. The maximum light intensity(S) in an area of 0° to (0.9×θm) was obtained with respect to the maximum emission angle θm of a fiber NA from the fiber NA. On the other hand, the maximum light intensity (N) in an area of 0.9×θm to 1.1×θm was obtained.

The S/N was calculated, and 1×10³ or more was determined to be acceptable.

### (6) Materials Used in Examples and Comparative Examples

Material A: Acrylic polymer (PMMA) (trade name “GH-1000S”, manufactured by Kuraray Co., Ltd.)

The refractive index measured after injection molding was 1.49.

Material B: Cycloolefin polymer (trade name “K26R”, manufactured by Zeon Corporation)

The refractive index measured after injection molding was 1.54.

Material C: Copolymer of 74.5 wt % of vinylidene fluoride/25.5 wt % of tetrafluoroethylene. The refractive index measured after injection molding was 1.40.

Material D: Copolymer of 18 wt % of vinylidene fluoride/62 wt % of tetrafluoroethylene/16 wt % of hexafluoropropylene/4 wt % of perfluoropropyl vinyl ether

The refractive index measured after injection molding was 1.35.

### (7) Dispersion of Carbon Black

Material E: The material A and carbon black were kneaded at a ratio of 96:4 (mass ratio) using a twin-screw extrusion melt kneader.

Material F: The material C and carbon black were kneaded at a ratio of 96:4 (mass ratio) using a twin-screw extrusion melt kneader.

Material G: The material C and the material F were mixed at a ratio of 39:1 (mass ratio) to obtain a carbon black content of 1000 ppm.

Material H: The material C and the material F were mixed at a ratio of 19:1 (mass ratio) to obtain a carbon black content of 2000 ppm.

Material I: The material C and the material F were mixed at a ratio of 9:1 (mass ratio) to obtain a carbon black content of 4000 ppm.

Material J: The material C and the material F were mixed at a ratio of 1:1 (mass ratio) to obtain a carbon black content of 20000 ppm.

Material K: The material A and the material E were mixed at a ratio of 19:1 (mass ratio) to obtain a carbon black content of 2000 ppm.

### (8) Content of Carbon Black

In these Examples, the carbon black content was calculated on the basis of the amount of use of raw materials. The above-described method is used in the case of measuring the carbon black content contained in the cladding using the optical fiber.

### (9) Measurement of Transmittance

Each material containing carbon black was processed into a 100 μm film shape with a hot press machine, and then a transmittance t100 at 650 nm was obtained using a spectrophotometer UV3510 (manufactured by Hitachi, Ltd.). From the transmittance t100, an absorbance A100 at the film thickness of 100 μm is obtained by the formula A=−LOG10 (t100). An absorbance A follows the Lambert-Beer's law that is proportional to an optical path length. For example, an absorbance A10 at 10 μm can be obtained as A10=A100/10 (An=A100/(100/n) when the thickness is n μm). From the absorbance A10, a transmittance t10 at the film thickness of 10 μm can be obtained by raising t10=10 to the power of −A10. The above-described method can be used in the case of measuring the transmittance of the cladding using the optical fiber.

### Example 1

The material A as the core material and the material H as the first cladding material were supplied to a conjugate spinning machine, and core-sheath conjugate melt spinning was performed at a temperature of 250° C. to obtain a plastic optical fiber 1 having a fiber diameter of 260 μm (core diameter: 240 μm, first cladding thickness: 10 μm) and a theoretical numerical aperture of 0.5. The transmittance obtained by forming the material H into a 100 μm film with a hot press machine was 4.8%. From this transmittance, the transmittance at the first cladding thickness of 10 μm was calculated to be 73.8%. The obtained plastic optical fiber had a transmission loss of 250 dB/km, which was favorable. The S/N ratio was 1.3×10⁴, which was favorable.

### Example 2

The material A as the core material and the material I as the first cladding material were supplied to a conjugate spinning machine, and core-sheath conjugate melt spinning was performed at a temperature of 250° C. to obtain a plastic optical fiber 2 having a fiber diameter of 260 μm (core diameter: 240 μm, first cladding thickness: 10 μm) and a theoretical numerical aperture of 0.5. The transmittance obtained by forming the material I into a 100 μm film with a hot press machine was 0.2%. From this transmittance, the transmittance at the first cladding thickness of 10 μm was calculated to be 54.5%. The obtained plastic optical fiber had a transmission loss of 450 dB/km, which was favorable. The S/N ratio was 3.5×10⁵, which was favorable.

### Example 3

The material A as the core material, the material H as the first cladding material, and the material A as the second cladding material were supplied to a conjugate spinning machine, and core-sheath conjugate melt spinning was performed at a temperature of 250° C. to obtain a plastic optical fiber 3 having a fiber diameter of 280 μm (core diameter: 240 μm, first cladding thickness: 10 μm, second cladding thickness: 10 μm) and a theoretical numerical aperture of 0.5. The transmittance obtained by forming the material H into a 100 μm film with a hot press machine was 4.8%. From this transmittance, the transmittance at the first cladding thickness of 10 μm was calculated to be 73.8%. The transmittance obtained by forming the material A into a 100 μm film with a hot press machine was 99.3%. From this transmittance, the transmittance at the second cladding thickness of 10 μm was calculated to be 99.9%. The obtained plastic optical fiber had a transmission loss of 250 dB/km, which was favorable. The S/N ratio was 7.5×10³, which was favorable.

### Example 4

The material A as the core material, the material H as the first cladding material, and the material K as the second cladding material were supplied to a conjugate spinning machine, and core-sheath conjugate melt spinning was performed at a temperature of 250° C. to obtain a plastic optical fiber having a fiber diameter of 280 μm (core diameter: 240 μm, first cladding thickness: 10 μm, second cladding thickness: 10 μm) and a theoretical numerical aperture of 0.5. The transmittance obtained by forming the material H into a 100 μm film with a hot press machine was 4.8%. From this transmittance, the transmittance at the first cladding thickness of 10 μm was calculated to be 73.8%. The transmittance obtained by forming the material K into a 100 μm film with a hot press machine was 20.0%. From this transmittance, the transmittance at the second cladding thickness of 10 μm was calculated to be 85.1%. The obtained plastic optical fiber had a transmission loss of 250 dB/km, which was favorable. The S/N ratio was 1.5×10⁶, which was favorable.

### Example 5

The material B as the core material and the material K as the first cladding material were supplied to a conjugate spinning machine, and core-sheath conjugate melt spinning was performed at a temperature of 250° C. to obtain a plastic optical fiber 5 having a fiber diameter of 260 μm (core diameter: 240 μm, first cladding thickness: 10 μm) and a theoretical numerical aperture of 0.3. The transmittance obtained by forming the material K into a 100 μm film with a hot press machine was 20.0%. From this transmittance, the transmittance at the first cladding thickness of 10 μm was calculated to be 85.1%. The obtained plastic optical fiber had a transmission loss of 650 dB/km, which was favorable. The S/N ratio was 5.3×10⁴, which was favorable.

### Example 6

The material A as the core material, the material G as the first cladding material, and the material K as the second cladding material were supplied to a conjugate spinning machine, and core-sheath conjugate melt spinning was performed at a temperature of 250° C. to obtain a plastic optical fiber having a fiber diameter of 280 μm (core diameter: 240 μm, first cladding thickness: 10 μm, second cladding thickness: 10 μm) and a theoretical numerical aperture of 0.5. The transmittance obtained by forming the material G into a 100 μm film with a hot press machine was 21.9%. From this transmittance, the transmittance at the first cladding thickness of 10 μm was calculated to be 85.9%. The transmittance obtained by forming the material K into a 100 μm film with a hot press machine was 20.0%. From this transmittance, the transmittance at the second cladding thickness of 10 μm was calculated to be 85.1%. The obtained plastic optical fiber had a transmission loss of 200 dB/km, which was best. The S/N ratio was 2.5×10⁶, which was favorable.

### Example 7

The material A as the core material, the material H as the first cladding material, and the material K as the second cladding material were supplied to a conjugate spinning machine, and core-sheath conjugate melt spinning was performed at a temperature of 250° C. to obtain a plastic optical fiber having a fiber diameter of 280 μm (core diameter: 240 μm, first cladding thickness: 5 μm, second cladding thickness: 15 μm) and a theoretical numerical aperture of 0.5. The transmittance obtained by forming the material H into a 100 μm film with a hot press machine was 4.8%. From this transmittance, the transmittance at the first cladding thickness of 5 μm was calculated to be 85.9%. The transmittance obtained by forming the material K into a 100 μm film with a hot press machine was 20.0%. From this transmittance, the transmittance at the second cladding thickness of 15 μm was calculated to be 78.6%. The obtained plastic optical fiber had a transmission loss of 250 dB/km, which was favorable. The S/N ratio was 7.5×10⁶, which was best.

### Comparative Example 1

The material A as the core material, the material C as the first cladding material, and the material D as the second cladding material were supplied to a conjugate spinning machine, and core-sheath conjugate melt spinning was performed at a temperature of 250° C. to obtain a plastic optical fiber 6 having a fiber diameter of 280 μm (core diameter: 240 μm, first cladding thickness: 10 μm, second cladding thickness: 10 μm) and a theoretical numerical aperture of 0.5. The transmittance obtained by forming the material C into a 100 μm film with a hot press machine was 98.0%. From this transmittance, the transmittance at the first cladding thickness of 10 μm was calculated to be 99.8%. The transmittance obtained by forming the material D into a 100 μm film with a hot press machine was 99.1%. From this transmittance, the transmittance at the second cladding thickness of 10 μm was calculated to be 99.9%. The obtained plastic optical fiber had a transmission loss of 200 dB/km, which was favorable. The S/N ratio was 2.6×10², which was unacceptable.

### Comparative Example 2

The material A as the core material, the material C as the first cladding material, and the material K as the second cladding material were supplied to a conjugate spinning machine, and core-sheath conjugate melt spinning was performed at a temperature of 250° C. to obtain a plastic optical fiber 7 having a fiber diameter of 280 μm (core diameter: 240 μm, first cladding thickness: 10 μm, second cladding thickness: 10 μm) and a theoretical numerical aperture of 0.5. The transmittance obtained by forming the material C into a 100 μm film with a hot press machine was 98.0%. From this transmittance, the transmittance at the first cladding thickness of 10 μm was calculated to be 99.8%. The transmittance obtained by forming the material K into a 100 μm film with a hot press machine was 20.0%. From this transmittance, the transmittance at the second cladding thickness of 10 μm was calculated to be 85.1%. The obtained plastic optical fiber had a transmission loss of 200 dB/km, which was favorable. The S/N ratio was 8.2×10², which was unacceptable.

### Comparative Example 3

The material B as the core material and the material D as the first cladding material were supplied to a conjugate spinning machine, and core-sheath conjugate melt spinning was performed at a temperature of 250° C. to obtain a plastic optical fiber 8 having a fiber diameter of 260 μm (core diameter: 240 μm, first cladding thickness: 10 μm) and a theoretical numerical aperture of 0.74. The transmittance obtained by forming the material D into a 100 μm film with a hot press machine was 99.1%. From this transmittance, the transmittance at the first cladding thickness of 10 μm was calculated to be 99.9%. The obtained plastic optical fiber had a transmission loss of 650 dB/km, which was favorable. The S/N ratio was 3.7×10¹, which was unacceptable.

### Comparative Example 4

The material B as the core material, the material A as the first cladding material, and the material H as the second cladding material were supplied to a conjugate spinning machine, and core-sheath conjugate melt spinning was performed at a temperature of 250° C. to obtain a plastic optical fiber 9 having a fiber diameter of 280 μm (core diameter: 240 μm, first cladding thickness: 10 μm, second cladding thickness: 10 μm) and a theoretical numerical aperture of 0.39. The transmittance obtained by forming the material A into a 100 μm film with a hot press machine was 99.3%. From this transmittance, the transmittance at the first cladding thickness of 10 μm was calculated to be 99.9%. The transmittance obtained by forming the material H into a 100 μm film with a hot press machine was 4.8%. From this transmittance, the transmittance at the second cladding thickness of 10 μm was calculated to be 73.8%. The obtained plastic optical fiber had a transmission loss of 670 dB/km, which was favorable. The S/N ratio was 5.4×10², which was unacceptable.

### Comparative Example 5

The material A as the core material and the material J as the first cladding material were supplied to a conjugate spinning machine, and core-sheath conjugate melt spinning was performed at a temperature of 250° C. to obtain a plastic optical fiber 10 having a fiber diameter of 260 μm (core diameter: 240 μm, first cladding thickness: 10 μm) and a theoretical numerical aperture of 0.5. The transmittance was measured by forming the material J into a 100 μm film with a hot press machine, but was the measurement lower limit. Therefore, since the transmittance at 100 μm of the material H and the transmittance at 10 μm of the material J are theoretically the same value, the transmittance of 4.8% at 100 μm of the material H was set as the transmittance at the first cladding thickness of 10 μm. The obtained plastic optical fiber had a transmission loss of 3000 dB/km, which was unacceptable. The S/N ratio was not able to be detected.

TABLE 1-1

| | | | | | First cladding | | | | | |
|---|---|---|---|---|---|---|---|---|---|---|
| | | | | | | | | | Trans- | |
| | | | | | | | Carbon | Trans- | mittance | |
| | Fiber | Core | | | | | addition | mittance | (%)/ | |
| | diameter | Diameter | | Refractive | Thickness | | amount | (%)/ | Cladding | Refractive |
| | (μm) | (μm) | Material | index | (μm) | Material | (ppm) | 100 μm | thickness | index |
| Example 1 | 260 | 240 | A | 1.49 | 10 | H | 2000 | 4.8 | 73.8 | 1.40 |
| Example 2 | 260 | 240 | A | 1.49 | 10 | I | 4000 | 0.2 | 54.5 | 1.40 |
| Example 3 | 280 | 240 | A | 1.49 | 10 | H | 2000 | 4.8 | 73.8 | 1.40 |
| Example 4 | 280 | 240 | A | 1.49 | 10 | H | 2000 | 4.8 | 73.8 | 1.40 |
| Example 5 | 260 | 240 | B | 1.54 | 10 | K | 2000 | 20.0 | 85.1 | 1.49 |
| Example 6 | 280 | 240 | A | 1.49 | 10 | G | 1000 | 21.9 | 85.9 | 1.40 |
| Example 7 | 280 | 240 | A | 1.49 | 5 | H | 2000 | 4.8 | 85.9 | 1.40 |
| Comparative | 280 | 240 | A | 1.49 | 10 | C | 0 | 98.0 | 99.8 | 1.40 |
| Example 1 | | | | | | | | | | |
| Comparative | 260 | 240 | A | 1.49 | 10 | C | 0 | 98.0 | 99.8 | 1.40 |
| Example 2 | | | | | | | | | | |
| Comparative | 260 | 240 | B | 1.54 | 10 | D | 0 | 99.1 | 99.9 | 1.35 |
| Example 3 | | | | | | | | | | |
| Comparative | 280 | 240 | B | 1.54 | 10 | A | 0 | 99.3 | 99.9 | 1.49 |
| Example 4 | | | | | | | | | | |
| Comparative | 260 | 240 | A | 1.49 | 10 | J | 20000 | Measurement | 4.8 | 1.40 |
| Example 5 | | | | | | | | lower limit | | |

| | | Second cladding | | | | | | | |
|---|---|---|---|---|---|---|---|---|---|
| | | | | | | Trans- | | | |
| | | | | Carbon | Trans- | mittance | | trans- | |
| | Numerical | | | addition | mittance | (%)/ | | mission | |
| | aperture | Thickness | | amount | (%)/ | Cladding | Refractive | loss | |
| | NA | (μm) | Material | (ppm) | 100 μm | thickness | index | (dB/km) | S/N ratio |
| Example 1 | 0.50 | — | — | — | — | — | — | 250 | 1.3 × 10E4 |
| Example 2 | 0.50 | — | — | — | — | — | — | 450 | 3.5 × 10E5 |
| Example 3 | 0.50 | 10 | A | 0 | 99.3 | 99.9 | 1.49 | 250 | 7.5 × 10E3 |
| Example 4 | 0.50 | 10 | K | 2000 | 20.0 | 85.1 | 1.49 | 250 | 1.5 × 10E6 |
| Example 5 | 0.30 | — | — | — | — | — | — | 650 | 5.3 × 10E4 |
| Example 6 | 0.50 | 10 | K | 2000 | 20.0 | 85.1 | 1.49 | 200 | 2.5 × 10E6 |
| Example 7 | 0.50 | 15 | K | 2000 | 20.0 | 78.6 | 1.49 | 250 | 7.5 × 10E6 |
| Comparative | 0.50 | 10 | D | 0 | 99.1 | 99.9 | 1.35 | 200 | 2.6 × 10E2 |
| Example 1 | | | | | | | | | |
| Comparative | 0.50 | 10 | K | 2000 | 20.0 | 85.1 | 1.49 | 200 | 8.2 × 10E2 |
| Example 2 | | | | | | | | | |
| Comparative | 0.74 | — | — | — | — | — | — | 650 | 3.7 × 10E1 |
| Example 3 | | | | | | | | | |
| Comparative | 0.39 | 10 | H | 2000 | 4.8 | 73.8 | 1.40 | 670 | 5.4 × 10E2 |
| Example 4 | | | | | | | | | |
| Comparative | 0.50 | — | — | — | — | — | — | 3000 | Undetectable |
| Example 5 | | | | | | | | | |

*In the table, 10En (n is a number) represents the n-th power of 10.

What is claimed is:

1. A plastic optical fiber comprising a core, a first cladding adjacent to the core and a second cladding outside the first cladding, wherein both of the first cladding and the second cladding contain a light-shielding agent, a content of the light-shielding agent contained in the first cladding is 100 to 10000 ppm, and
transmittance t1 of light having a wavelength of 650 nm at a film thickness of the first cladding and a transmittance t2 of light having a wavelength of 650 nm at a film thickness of the second cladding have a relationship of t1>t2.

2. The plastic optical fiber according to claim 1, wherein the light-shielding agent contained in the first cladding is carbon black.

3. (canceled)

4. The plastic optical fiber according to claim 1, wherein a refractive index of the second cladding is larger than a refractive index of the first cladding.

5. The plastic optical fiber according to claim 1, wherein a content of the light-shielding agent contained in the second cladding is 100 to 10000 ppm.

6. (canceled)

7. The plastic optical fiber according to claim 5, wherein the light-shielding agent contained in the second cladding is carbon black.

8. The plastic optical fiber according to claim 5, wherein the light-shielding agent contained in the first cladding and the light-shielding agent contained in the second cladding are made of a same material, and a light-shielding agent content D1 of the first cladding and a light-shielding agent content D2 of the second cladding have a relationship of D1<D2.

9. The plastic optical fiber according to claim 1, wherein a plastic optical fiber forming film thickness T1 of the first cladding and a plastic optical fiber forming film thickness T2 of the second cladding have a relationship of T1<T2.

10. The plastic optical fiber according to claim 1, wherein the core is made of polymethyl methacrylate, and a numerical aperture NA of the core and the first cladding is 0.65 or less.

11. A medical sensor device comprising the plastic optical fiber according to claim 1.

12. The plastic optical fiber according to claim 1, wherein a transmittance t1 of light having a wavelength of 650 nm at a film thickness of the first cladding and a transmittance t2 of light having a wavelength of 650 nm at a film thickness of the second cladding have a relationship of t1>t2.

13. The plastic optical fiber according to claim 1, wherein the light-shielding agent contained in the second cladding is carbon black.

14. The plastic optical fiber according to claim 1, wherein the light-shielding agent contained in the first cladding and the light-shielding agent contained in the second cladding are made of a same material, and a light-shielding agent content D1 of the first cladding and a light-shielding agent content D2 of the second cladding have a relationship of D1<D2.

15. The plastic optical fiber according to claim 1, wherein a plastic optical fiber forming film thickness T1 of the first cladding and a plastic optical fiber forming film thickness T2 of the second cladding have a relationship of T1<T2.
